(19) United States
(12) Patent Application Publication
(10) Pub. No.: US 2021/0052255 A1
(43) Pub. Date: Feb. 25, 2021

(54) ULTRASOUND GUIDANCE DYNAMIC MODE SWITCHING

(71) Applicant: Bay Labs, Inc., San Francisco, CA (US)

(72) Inventors: Charles Cadieu, Menlo Park, CA (US); Michael G. Cannon, Haverford, PA (US); Ali Chaudhry, San Francisco, CA (US); Ha Hong, San Ramon, CA (US); Kilian Koepsell, San Francisco, CA (US); Nripesh Parajuli, Millbrae, CA (US); Nicolas Poilvert, Seattle, WA (US)

(21) Appl. No.: 16/544,657

(22) Filed: Aug. 19, 2019

(51) Int. Cl. A61B 8/00; A61B 8/08

(52) U.S. Cl. CPC A61B 8/54; A61B 8/465; A61B 8/13; A61B 8/0883; A61B 8/463; A61B 8/467

(57) ABSTRACT

An ultrasound guidance dynamic mode switching method includes selecting a predetermined ultrasound diagnostic procedure in memory of an ultrasound diagnostic computing system and identifying an operating mode of the system for a first sequence of views stored in the memory as a workflow in correspondence to the selected procedure. The method additionally includes placing the system in the identified operating mode and acquiring imagery of a target organ in association with the views of the first sequence of the workflow. Finally, the method includes detecting in the acquired imagery, a feature of the target organ mapped to a different operating mode, and in response to the detection, displaying a recommendation in a display to change operating modes, placing the system into the different operating mode, and acquiring additional imagery of the target organ utilizing the system in association with a different sequence of additional views of a different workflow.

### BACKGROUND OF THE INVENTION

### Field of the Invention

The present invention relates to ultrasound imaging and more particularly to ultrasound image acquisition.

### Description of the Related Art

Medical imaging refers to the process of creating a visual representation of an interior portion of a mammalian body for the purpose of clinical analysis and medical intervention. Medical imaging seeks to reveal internal structures hidden by the exterior of the body so as to facilitate the diagnosis and treatment of disease. Medical imaging incorporates several different image acquisition methodologies and corresponding radiological devices technologies. Common techniques include X-ray radiography including computerized tomography (CT), magnetic resonance imaging (MRI), medical ultrasonography or ultrasound, endoscopy, elastography, tactile imaging, thermography, medical photography and nuclear medicine functional imaging techniques as positron emission tomography (PET) and Single-photon emission computed tomography (SPECT). Depending upon the desired use of the imagery for the purpose of a medical diagnosis or the targeting of specific tissue or a particular organ or portion of an organ, different techniques and devices for different imagery may be preferred.

Ultrasound imaging, also known as sonography, is a medical imaging technique that employs high-frequency sound waves to view three-dimensional structures inside the body of a living being. Because ultrasound images are captured in real-time, ultrasound images also show movement of the internal organs of the body as well as blood flowing through the blood vessels of the human body and the stiffness of tissue. Unlike x-ray imaging, ultrasound imaging does not involve ionizing radiation thereby allowing prolonged usage of ultrasound imaging without threatening tissue and internal organ damage from prolonged radiation exposure.

To acquire ultrasound imagery, during an ultrasound exam, a transducer, commonly referred to as a probe, is placed directly on the skin or inside a body opening. The probe is coupled to image generation circuitry that includes circuitry adapted to transmit and receive signals to and from the probe, and may include a beamformer, though synthetic aperture imaging systems may use retrospective image formation reducing the need for beamforming and scan conversion functions. A thin layer of gel is applied to the skin so that the ultrasound waves are transmitted from the transducer through the medium of the gel into the body. The ultrasound image is produced based upon a measurement of the reflection of the ultrasound waves off the body structures. The strength of the ultrasound signal, measured as the amplitude of the detected sound wave reflection, and the time taken for the sound wave to travel through the body provide the information necessary to compute an image of target structures of the body. The “Doppler” effect may be used in ultrasound imagery to measure the velocity and direction of fluid flow within the structures of the body (namely, blood).

Ultrasound allows multiple types of scanning modes. These modes interrogate the target using different transducer pulsing and image generation techniques in order to visualize anatomy and function for various clinical purposes. For example, two-dimensional imaging provides a two-dimensional visualization of structures. Color Doppler ultrasound imaging provides a color map of blood flow combined with a two-dimensional image. Pulsed Wave and Continuous Wave Doppler ultrasound imaging provide a spectral histogram of blood flow velocity and amplitude. Strain imaging visualizes tissue elasticity. Three-dimensional mode visualizes structure and blood flow in three dimensions. The use of different scanning modes is important in making a complete diagnosis of health conditions.

Compared to other prominent methods of medical imaging, ultrasound presents several advantages to the diagnostician and patient. First and foremost, ultrasound imaging provides images in real-time. As well, ultrasound imaging requires equipment that is portable and can be brought to the bedside of the patient. Further, as a practical matter, the ultrasound imaging equipment is substantially lower in cost than other medical imaging equipment, and as noted, does not use harmful ionizing radiation. Even still, ultrasound imagery is not without challenge.

For example, in some instances, an attempted view of a target organ may be incomplete omitting key features of the target organ from the view due to anatomical limitations or an improper placement of the imaging sensor. In this regard, as to the term “view”, the ultrasound imaging of a target area of the body may be achieved from many different “views” utilizing the ultrasound probe. Each view may be achieved through a combination of position and pose of the probe such that the angle and approach of the ultrasound probe generally results in a different perspective “view” of the target area. Generally, a particular view of the target area presented in an ultrasound image may be preferred depending upon the desired use of the imagery for the purpose of a medical diagnosis or the targeting of specific tissue or a particular organ or portion thereof. More to the point, different views of the same target area produce imagery with emphasis on different anatomical features such that some views are known to have the highest probability of producing imagery of a feature of interest. As well, different views can also be required in order to perform measurements that are used for diagnostic purposes.

Thus, depending upon the particular feature of interest, the operator must first know the desired view to best image the feature of interest and then, with respect to the portion of the body selected for imaging and the desired view, the skilled operator must know where to initially place the ultrasound probe on the body. Then, the skilled operator must know how to spatially orient the probe and finally, the skilled operator must know where to move the probe so as to acquire the desired imagery. Generally, the ultrasound operator is guided in the initial placement, orientation and movement of the probe based upon the visual feedback provided by the imagery produced during the ultrasound. Thus, essentially, the navigation of the probe is a manual process consisting of iterative trial and error and requires specialized knowledge and expertise on the part of the ultrasound operator—especially in the selection of a route of views through which the probe must to produce a complete exam.

Importantly, given the nature of conventional ultrasound imaging, the resultant images of a target area of the body may vary in quality. That is to say, depending upon the operator, the clarity and focal point of a medical image may vary. As well, external factors such as the anatomical features of the body may inhibit clarity of key features of the target organ despite proper placement of the imaging sensor. Yet, whereas certain anatomical features may inhibit a quality image of a target area in one view, a different view of the same target area or even a slightly different target area may provide higher quality imagery of the anatomical feature sought for imaging by the practitioner. As can be seen, then, the production of quality ultrasound images remains highly dependent upon a skilled operator.

### BRIEF SUMMARY OF THE INVENTION

Embodiments of the present invention address deficiencies of the art in respect to ultrasound imaging and provide a novel and non-obvious method, system and computer program product for ultrasound guidance dynamic mode switching. In an embodiment of the invention, an ultrasound guidance dynamic mode switching method includes selecting a predetermined ultrasound diagnostic procedure in memory of an ultrasound diagnostic computing system and identifying an operating mode of the ultrasound diagnostic computing system for a first sequence of views stored in the memory as a workflow in correspondence to the selected procedure. The method additionally includes placing the ultrasound diagnostic computing system in the identified operating mode and acquiring imagery of a target organ utilizing the computing system in association with the views of the first sequence of the workflow. Finally, the method includes detecting in the acquired imagery, a feature of the target organ mapped to a different operating mode of the ultrasound diagnostic computing system, and in response to the detection, displaying a recommendation in a display of the ultrasound diagnostic computing system to change operating modes of the ultrasound diagnostic computing system, placing the ultrasound diagnostic computing system into the different operating mode mapped to the feature, and acquiring additional imagery of the target organ utilizing the ultrasound diagnostic computing system in association with a different sequence of additional views of a different workflow.

In one aspect of the embodiment, the method additionally includes identifying a measurement to be performed based upon detecting in the acquired imagery, a feature of the target organ that requires a measurement using a different one of the operating modes of the ultrasound diagnostic computing system. In response, a different one of the operating modes is identified in association with the identified measurement. The different one of the operating modes is then presented as a substitute for a contemporaneous one of the operating modes in order to perform the identified measurement. An exemplary measurement includes a measurement of fluid velocity—namely blood velocity in proximity to the target organ.

In one aspect of the embodiment, the identified operating mode is either a two-dimensional ultrasound mode or a three-dimensional ultrasound mode. In another aspect of the embodiment, the different operating mode is a non-imaging continuous wave (CW) ultrasound mode. In yet another aspect of the embodiment, the different operating mode is a Doppler ultrasound mode, such as color flow Doppler ultrasound mode, pulsed wave Doppler ultrasound mode, continuous wave Doppler ultrasound mode or Doppler tissue imaging ultrasound mode. Another possible operating mode is strain imaging ultrasound mode. In even yet another aspect of the embodiment, the target organ is a heart and the feature is a stenotic valve velocity that exceeds a threshold rate. Finally, in even yet another aspect of the embodiment, the method additionally includes annotating a digital file storing the acquired additional imagery with a textual reference to the recommended change of operating mode.

In another embodiment of the invention, a data processing system is configured for ultrasound guidance dynamic mode switching. The system includes a computer with memory and at least one processor, a display coupled to the computer; image generation circuitry coupled to the computer and the display and an ultrasound imaging probe comprising a transducer connected to the image generation circuitry. The system further includes an ultrasound guidance dynamic progression module executing in the memory of the computer. The module includes program code enabled upon execution by the processor of the computer to select a predetermined ultrasound diagnostic procedure in the memory of the computer, identify an operating mode of the ultrasound imaging probe for a first sequence of views stored in the memory as a workflow in correspondence to the selected procedure, place the ultrasound imaging probe in the identified operating mode and acquire imagery of a target organ utilizing the ultrasound imaging probe in association with the views of the first sequence of the workflow.

Of import, the program code further is enabled to detect in the acquired imagery, a feature of the target organ mapped to a different operating mode of the ultrasound imaging probe and to respond to the detection by displaying a recommendation in a display of the computer to change operating modes of the ultrasound imaging probe, placing the ultrasound imaging probe into the different operating mode mapped to the feature, and acquiring additional imagery of the target organ utilizing the ultrasound imaging probe in association with a different sequence of additional views of a different workflow.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is pictorial illustration of a process for ultrasound guidance dynamic mode switching;

FIG. 2 is a schematic illustration of an ultrasound diagnostics data processing system configured for ultrasound guidance dynamic mode switching; and,

FIG. 3 is a flow chart illustrating a process for ultrasound guidance dynamic mode switching.

### DETAILED DESCRIPTION OF THE INVENTION

Embodiments of the invention provide for ultrasound guidance dynamic mode switching. In accordance with an embodiment of the invention, an ultrasound diagnostic computing system is placed into a first operating mode in which imagery of a target organ is acquired according to a sequence of views in a workflow. During the acquisition of the imagery, a feature may be detected within the acquired imagery. The feature is then mapped to a different operating mode of the ultrasound diagnostic computing system. As such, in response to the detection of the feature, a recommendation is displayed in the ultrasound diagnostics computing system to change the operating mode of the ultrasound diagnostics computing system to the different operating mode mapped to the feature, including changing a transducer used in connection with the acquisition of ultrasound imagery. Then, the ultrasound diagnostic computing system is placed into the different operating mode and additional imagery of the target organ acquired in association with a different sequence of additional views of a different workflow.

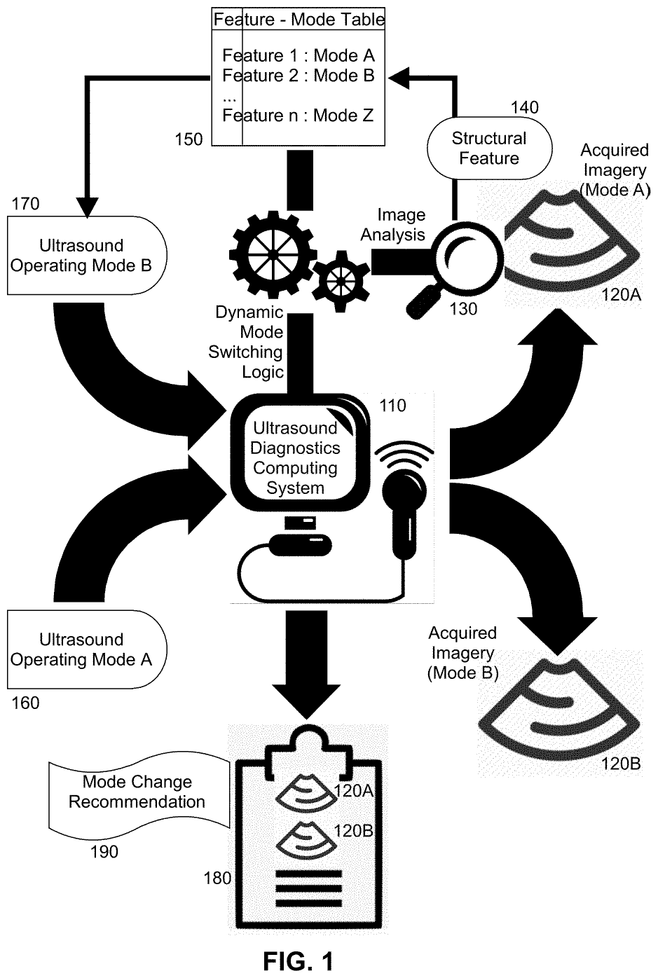

In further illustration, FIG. 1 pictorially shows a process for ultrasound guidance dynamic mode switching. As shown in FIG. 1, an ultrasound diagnostics computing system 110 is placed in an initial operating mode 160, for instance a two-dimensional imaging mode such as an ultrasound image acquisition B-mode. Thereafter, the ultrasound diagnostics computing system 110 acquires initial imagery 120A of a target organ according to the initial operating mode 160. An image analysis portion 130 of dynamic mode switching logic 100 processes the acquired initial imagery 120A to detect a particular feature 140 within the initial imagery 120A. In this regard, the feature 140 may include a prediction of a disease state of the target organ for example blood flow characteristics of the target organ, and the image analysis portion 130 may include a convolutional neural network trained to characterize imagery as containing specific features including the particular feature 140.

Upon detecting the feature 140, the dynamic mode switching logic 100 maps the feature 140 to a corresponding operating mode of the ultrasound diagnostics computing system 110. For example, the dynamic mode switching logic 100 can consult a table 150 to identify the recommended operating mode 170 in response to which the dynamic mode switching logic 100 presents in a display of the ultrasound diagnostics computing system 110, an instruction to switch operating modes from the initial operating mode 160 to the recommended operating mode 170, for instance a non-imaging CW transducer image acquisition mode. The table 150 can identify a measurement that should be performed which requires a different operating mode, and recommend the mode switch in order that images for performing the measurement are produced. Thereafter, additional imagery 120B is acquired in accordance with the recommended operating mode 170.

Once the additional imagery 120B has been acquired, both the acquired initial imagery 120A and the additional imagery 120B are included in an ultrasound diagnostics report 180 along with an annotation 190 of the recommendation to change modes from the initial operating mode 160 to the recommended operating mode 170. Even further, to the extent that the image analysis portion 130 of the dynamic mode switching logic 100 is enabled to detect a specific mode from which the acquired imagery 120A, 120B had been acquired, the detected modes are further included in the report 180 as part of the annotation 190. Finally, to the extent that the image analysis portion 130 of the dynamic mode switching logic 100 is enabled to detect a specific view from which the acquired imagery 120A, 120B had been acquired, the specific views are yet further included in the report 180 as part of the annotation 190. In this way, a diagnostician reviewing the report 190 will have the confidence that the requisite switch in modes occurred in consequence of the feature 140 detected in the initial imagery 120A and that the additional imagery 120B had been acquired utilizing the recommended mode 170 with appropriate views.

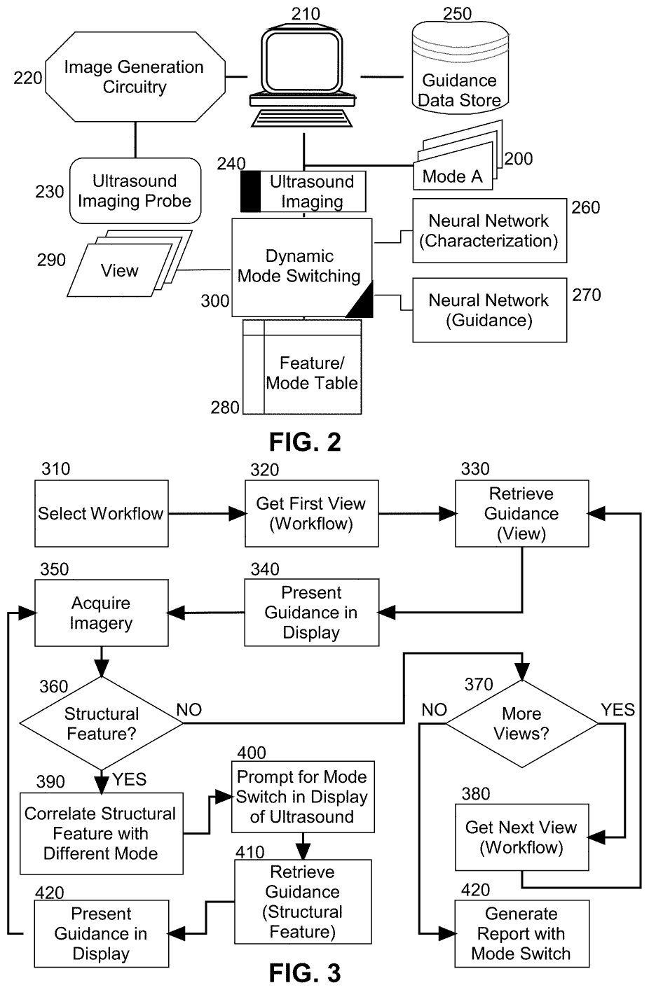

The process described in connection with FIG. 1 may be implemented in an ultrasound diagnostics data processing system. In further illustration, FIG. 2 schematically shows an ultrasound diagnostics data processing system configured for ultrasound guidance dynamic mode switching. The system includes a host computing system 210 that includes a computer with at least one processor, memory and a display. The host computing system 210 also includes a data store 250. The host computing system 210 yet further is coupled to an ultrasound imaging system 240 adapted to store in memory, ultrasound imagery acquired through the placement of an imaging transducer 230 proximate to a target organ of interest in a mammalian subject by operation of image generation circuitry 220. In this regard, the imaging transducer 230 can include a multi-mode phased array transducer, or a non-imaging CW doppler mode transducer, to name two examples. As such, the ultrasound imaging system 240 can operate in one of a multiplicity of modes 200, such as those associated with a multi-mode phased array transducer—two-dimensional ultrasound mode, three-dimensional ultrasound mode, Doppler ultrasound mode, color flow mapping mode, pulsed wave (PW) mode and CW mode, also associated with the non-imaging CW doppler mode transducer.

The host computing system 210 is communicatively coupled to fixed storage (not shown), either locally or remotely (“in the cloud”) storing therein one or more neural networks 260 and a programmatic interface to the neural networks 260. The neural network 260 is trained to characterize one or more features of the target organ, for example one or more physical components of the target organ, or the physical performance of the target organ. To do so, imagery of a specified view of the target organ acquired by the ultrasound imaging system 240 is provided to the neural network 260 which in turn accesses the programmatic interface so that the neural network 260 may then output the characterization for the imagery along with an indication of confidence in that characterization. The ultrasound imaging system 240 in turn renders on the display of the host computing system 210 not only the imagery, but also the characterization and optionally, the indication of confidence in that characterization.

As well, a second neural network 270 may be trained to characterize guidance instructions relative to contemporaneously acquired imagery of the target organ. In this regard, with respect to a particular one of the views 290, the second neural network 270 is trained to produce recommend guidance to achieve the optimal acquisition of imagery for the target organ for the particular one of the views 290, relative to the imagery contemporaneously presented in a display of the host computing system 210. For example, in connection with the imaging of a heart, the views 290 may include a parasternal long axis view, a parasternal short axis view, an apical two, three, four or five chamber view or a subcoastal view, to name a few examples. To that end, as the neural network 270 is presented with contemporaneously acquired imagery of the target organ for the particular one of the views 290, the neural network produces a recommended movement or pose of the ultrasound imaging transducer 230 in order to acquire imagery deemed acceptable for the particular one of the views 290.

Importantly, a dynamic mode switching module 300 is coupled to the ultrasound imaging system 240. The dynamic mode switching module 300 includes computer program instructions that when executing in the memory of the host computing system 210, are enabled to group together a sequence of the different views 290 as a workflow, place the ultrasound imaging system 240 into a specified one of the operating modes 200, and, for each of the views 290 in the sequence, retrieve from the data store 250 guidance instructions necessary to optimally acquire imagery for the selected one of the views 290 according to the specified one of the operating modes 200 utilizing a correspondingly appropriate ultrasound imaging transducer 230.

The program instructions are further enabled to receive from the neural network 260 in characterizing acquired real-time imagery for a selected one of the views 250 of a workflow, an indication of a feature of interest, such as the presence of a component of the target organ indicative of disease, or the visualized performance of the target organ indicative of disease. Examples include a threshold flow rate of blood through a portion of the target organ. In the context of the heart, for example, velocity of blood through a valve can be an indication of valvular stenosis.

The program instructions of the module 300 then are adapted to correlate the feature with a different one of the operating modes 200 in a table 280 and a selection of one or more of the views 290. As well, the program instructions of the module 300 are enabled to retrieve guidance from the data store 250 for the correlated one of the views 290, and to present a prompt in the display of the host computing system 210 recommending a change in operating mode including, the selection of a different ultrasound imaging transducer. Finally, once the program instructions are enabled to display the retrieved guidance in the display of the host computing system 210 so as to facilitate the acquisition of additional imagery of the target organ utilizing the different ultrasound imaging transducer in the recommended one of the modes 200.

In even yet further illustration of the operation of the dynamic mode switching module 300, FIG. 3 is a flow chart illustrating a process for ultrasound guidance dynamic mode switching. Beginning in block 310, a workflow is selected including a multiplicity of different views of a target organ. In block 320, a first one of the views is selected for acquiring imagery of the target organ and in block 330, guidance instructions for the first view are retrieved into memory for display in a user interface to the ultrasound imaging system. In this regard, the guidance instructions may be retrieved from a fixed data store irrespective of any contemporaneous imagery acquired for the first view, or the guidance instructions may be selected based upon the output of a neural network trained to produce guidance instructions for a particular view based upon the content of contemporaneous imagery acquired for the first view. In either circumstance, in block 340, the guidance instructions are displayed in the user interface to the ultrasound imaging system.

In block 350, imagery is acquired in the ultrasound imaging system for the target organ utilizing a first imaging transducer with the ultrasound imaging system having been placed in an initial operating mode. In decision block 360, it is determined whether or not a particular feature exists in connection with the acquired real-time imagery. For instance, the real-time imagery may be submitted to a neural network trained to classify features such as the presence of a physical component of the target organ indicative of disease, or the physical operation of the target organ indicative of disease. Examples include detecting a state of a valve of the heart indicative of disease, or a threshold velocity of blood passing through the valve indicative of disease. If the particular feature is determined not to exist in the acquired imagery, in decision block 370 it is determined if additional views remain to be processed in the workflow. If so, a next view of the workflow is selected and the process repeats in block 330. Otherwise, the process proceeds to block 390.

In block 390, responsive to a determination that the particular feature has been detected in connection with the acquired imagery, the feature is correlated with a different operating mode and a set of one or more views. In block 400, a prompt is generated in the user interface for the ultrasound imaging system to change operating modes to a different operating mode and optionally, to change imaging transducers. Thereafter, in block 410 guidance for a first view associated with the different operating mode is retrieved and in block 420 the guidance for the first view of the different operating mode is presented in the user interface for the ultrasound imaging system. Finally, the process returns to decision block 350 in which new real-time imagery is acquired utilizing the different operating mode and optionally, the different imaging transducer, and in decision block 360 it is determined if additional features are detected in the newly acquired imagery.

Thereafter, in decision block 370, if it is determined that no additional views remain to be processed in the workflow, a report is generated including the acquired imagery, with each image annotated to indicate a corresponding view and operating mode utilized to acquire the image, and also an indication of the recommendation to change operating modes. In this way, a diagnostician reviewing the report will recognize not only the detection of the feature giving rise to the recommendation to switch operating modes, and optionally imaging transducers, but also an assurance that the operating mode had been switched in order to acquire the additional imagery utilizing the recommended views utilizing the recommended imaging transducer.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. An ultrasound guidance dynamic mode switching method comprising:
selecting a predetermined ultrasound diagnostic procedure in memory of an ultrasound diagnostic computing system;
identifying an operating mode of the ultrasound diagnostic computing system for a first sequence of views stored in the memory as a workflow in correspondence to the selected procedure;
placing the ultrasound diagnostic computing system in the identified operating mode and acquiring imagery of a target organ utilizing the computing system in association with the views of the first sequence of the workflow;
detecting in the acquired imagery, a feature of the target organ mapped to a different operating mode of the ultrasound diagnostic computing system; and,
responsive to the detection, displaying a recommendation in a display of the ultrasound diagnostic computing system to change operating modes of the ultrasound diagnostic computing system, placing the ultrasound diagnostic computing system into the different operating mode mapped to the feature, and acquiring additional imagery of the target organ utilizing the ultrasound diagnostic computing system in association with a different sequence of additional views of a different workflow.

2. The method of claim 1, wherein displaying a recommendation in a display of the ultrasound diagnostic computing system to change operating modes, comprises:
identifying a measurement to be performed based upon detecting in the acquired imagery, a feature of the target organ that requires a measurement using a different operating mode of the ultrasound diagnostic computing system; and,
selecting the different operating mode as a substitute for the identified operating mode in order to perform the measurement; and,
displaying a recommendation to change to the different operating mode in the display of the ultrasound diagnostic computing system.

3. The method of claim 1, wherein the identified operating mode is an operating mode selected from the group consisting of a two-dimensional ultrasound mode and a three-dimensional operating mode.

4. The method of claim 1, wherein the different operating mode is a non-imaging continuous wave (CW) ultrasound mode.

5. The method of claim 1, wherein the different operating mode is a Doppler ultrasound mode.

6. The method of claim 1, wherein the target organ is a heart and the feature is a stenotic valve velocity that exceeds a threshold rate.

7. The method of claim 1, further comprising annotating a digital file storing the acquired additional imagery with a textual reference to the recommended change of operating mode.

8. A data processing system configured for ultrasound guidance dynamic mode switching method comprising:
a computer with memory and at least one processor;
a display coupled to the computer;
image generation circuitry coupled to the computer and the display;
an ultrasound imaging probe comprising a transducer connected to the image generation circuitry; and,
an ultrasound guidance dynamic progression module executing in the memory of the computer, the module comprising program code enabled upon execution by the processor of the computer to perform:
selecting a predetermined ultrasound diagnostic procedure in the memory of the computer;
identifying an operating mode of the ultrasound imaging probe for a first sequence of views stored in the memory as a workflow in correspondence to the selected procedure;
placing the ultrasound imaging probe in the identified operating mode and acquiring imagery of a target organ utilizing the ultrasound imaging probe in association with the views of the first sequence of the workflow;
detecting in the acquired imagery, a feature of the target organ mapped to a different operating mode of the ultrasound imaging probe; and,
responsive to the detection, displaying a recommendation in a display of the computer to change operating modes of the ultrasound imaging probe, placing the ultrasound imaging probe into the different operating mode mapped to the feature, and acquiring additional imagery of the target organ utilizing the ultrasound imaging probe in association with a different sequence of additional views of a different workflow.

9. The system of claim 8, wherein displaying a recommendation in a display of the ultrasound diagnostic computing system to change operating modes, comprises:
identifying a measurement to be performed based upon detecting in the acquired imagery, a feature of the target organ that requires a measurement using a different operating mode of the ultrasound diagnostic computing system; and,
selecting the different operating mode as a substitute for the identified operating mode in order to perform the measurement; and,
displaying a recommendation to change to the different operating mode in the display of the ultrasound diagnostic computing system.

10. The system of claim 8, wherein the identified operating mode is an operating mode selected from the group consisting of a two-dimensional ultrasound mode and a three-dimensional ultrasound mode.

11. The system of claim 8, wherein the different operating mode is a non-imaging continuous wave (CW) ultrasound mode.

12. The system of claim 8, wherein the different operating mode is a Doppler ultrasound mode.

13. The system of claim 8, wherein the target organ is a heart and the (delete?) feature is a stenotic valve velocity that exceeds a threshold rate.

14. The system of claim 8, further comprising annotating a digital file storing the acquired additional imagery with a textual reference to the recommended change of operating mode.

15. A computer program product for ultrasound guidance dynamic mode switching, the computer program product including a computer readable storage medium having program instructions embodied therewith, the program instructions executable by a device to cause the device to perform a method including:
selecting a predetermined ultrasound diagnostic procedure in memory of an ultrasound diagnostic computing system;
identifying an operating mode of the ultrasound diagnostic computing system for a first sequence of views stored in the memory as a workflow in correspondence to the selected procedure;
placing the ultrasound diagnostic computing system in the identified operating mode and acquiring imagery of a target organ utilizing the computing system in association with the views of the first sequence of the workflow;
detecting in the acquired imagery, a feature of the target organ mapped to a different operating mode of the ultrasound diagnostic computing system; and,
responsive to the detection, displaying a recommendation in a display of the ultrasound diagnostic computing system to change operating modes of the ultrasound diagnostic computing system, placing the ultrasound diagnostic computing system into the different operating mode mapped to the feature, and acquiring additional imagery of the target organ utilizing the ultrasound diagnostic computing system in association with a different sequence of additional views of a different workflow.

16. The computer program product of claim 15, wherein displaying a recommendation in a display of the ultrasound diagnostic computing system to change operating modes, comprises:
identifying a measurement to be performed based upon detecting in the acquired imagery, a feature of the target organ that requires a measurement using a different operating mode of the ultrasound diagnostic computing system; and,
selecting the different operating mode as a substitute for the identified operating mode in order to perform the measurement; and,
displaying a recommendation to change to the different operating mode in the display of the ultrasound diagnostic computing system.

17. The computer program product of claim 15, wherein the identified operating mode is an operating mode selected from the group consisting of a two-dimensional ultrasound mode and a three-dimensional ultrasound mode.

18. The computer program product of claim 15, wherein the different operating mode is a non-imaging continuous wave (CW) ultrasound mode.

19. The computer program product of claim 15, wherein the different operating mode is a Doppler ultrasound mode.

20. The computer program product of claim 15, wherein the target organ is a heart and the feature is a stenotic valve velocity that exceeds a threshold rate.
